(19) United States
(12) Patent Application Publication
(10) Pub. No.: US 2026/0240968 A1
(43) Pub. Date: Aug. 20, 2026

(54) METHODS AND MODIFICATIONS FOR REDUCING INNATE IMMUNE RESPONSES TO RNA

(71) Applicant: The Board of Trustees of the Leland Stanford Junior University, Stanford, CA (US)

(72) Inventors: Eric T. Kool, Stanford, CA (US); Linglan Fang, Stanford, CA (US)

(21) Appl. No.: 19/152,861

(22) PCT Filed: Feb. 5, 2024
(86) PCT No.: PCT/US2024/014445

§ 371 Date: Jul. 31, 2025

Related U.S. Application Data
(60) Provisional application No. 63/443,651, filed on Feb. 6, 2023.

(51) Int. Cl. A61K 39/00; A61K 31/7115; A61K 31/712; A61K 38/16; A61K 39/385; A61K 48/00; C12N 15/11

(52) U.S. Cl. CPC A61K 39/0011; A61K 31/7115; A61K 31/712; A61K 38/16; A61K 39/385; A61K 48/005; C12N 15/11; A61K 2039/53

(57) ABSTRACT

Compositions and methods are provided for reducing RNA interactions with cellular RNA sensors, including without limitation toll-like receptors (TLRs), RIG-I and MDA-5, by cloaking the RNA using post-transcriptional, selective 2′-hydroxyl post-synthetic modification. The post-synthetic modification (referred to as “cloaking”) of 2′-hydroxyl groups of RNA is optionally spontaneously reversed in cells, restoring biologically functional RNA in cells. This RNA modification reduces the innate immunogenicity of the RNA while allowing for other biological activities, such as translation, to be retained.

### CROSS REFERENCE TO OTHER APPLICATIONS

This application claims the benefit of U.S. Provisional Application No. 63/443,651, filed Feb. 6, 2023, the contents of which are hereby incorporated by reference in its entirety.

### STATEMENT REGARDING FEDERALLY SPONSORED RESEARCH OR DEVELOPMENT

This invention was made with Government support under contract GM145357 awarded by the National Institutes of Health. The Government has certain rights in the invention.

### BACKGROUND

RNA has a number of uses as a therapeutic agent. For example, mRNA vaccines are used, or are in development, for immunization against viruses, cancers, and the like. The recent development and rapid distribution of mRNA-based SARS-COVID-19 vaccines have demonstrated the potential of mRNA for vaccination purposes. RNAs are also in development for use in siRNAs, circRNAs, miRNA sponges, CRISPR technologies, and base editing technologies.

However, RNA delivered to cells can also overstimulate the human innate immune system by activation of cellular RNA sensors, triggering the cellular antiviral defense mechanism that leads to inhibited mRNA translation and systemic inflammation in humans. For example, RNA can be recognized in human cells by TLR3, TLR7, TLR8, RIG-1 and MDA-5, in a sequence- and structure-dependent manner. The resultant activation of the innate immune system is cell specific and tissue specific. Unmodified mRNA synthesized by in vitro transcription is a potent inducer of the production of type I interferons mediated by TLR3, TLR7, TLR8 and RIG-I, as well as pro-inflammatory cytokines and chemokines, which have been found to hamper the translation efficiency of the encoded antigen protein and can lead to other undesired physiological effects such as pain.

Multiple approaches have been deployed to minimize innate immune response towards mRNA, including RNA sequence optimization, advanced purification and RNA delivery methods, and incorporation of the naturally modified nucleobases. For example, modified nucleobases (e.g., m5C, pseudouridine, 1-methylpseudouridine) can reduce such immunogenic activity by disrupting RNA interactions with cellular receptors/RNA sensors.

Chemically modifying nucleosides at multiple 2′-OH groups of RNA via post-synthetic modification (referred to as “cloaking”) for therapeutic indications is of interest, particularly if the modification can be reversed to provide for biologically active RNA molecules. The present disclosure provides such protection and methods for modulating the effects of RNA on innate immunity.

### SUMMARY

Compositions and methods are provided for reducing RNA interactions with cellular RNA sensors, including without limitation toll-like receptors (TLRs), RIG-1 and MDA-5, by cloaking the RNA using post-transcriptional, selective 2′-hydroxyl acylation of RNA by reaction with a cloaking reagent such as water-soluble acylimidazole, sulfonylimidazole, sulfonyltriazole, and related activated acyl reagents, as disclosed herein. The 2′-acylation is optionally spontaneously reversed in cells, restoring biologically functional RNA in cells. This RNA modification reduces the innate immunogenicity of the RNA while allowing for other biological activities, such as translation, to be retained.

The RNA that is cloaked may be mRNA, tRNA, rRNA, circRNA, RNA sponges, long noncoding RNA, viral RNA, synthetic RNA such as chemically synthesized or in vitro transcribed forms, or any other form of RNA, such as hnRNA and viroid RNA. In some embodiments the RNA is in vitro transcribed mRNA. In some embodiments an mRNA encodes a protein, e.g. an antigen, a therapeutic protein, a growth factor, a structural protein, etc. An antigen may be, for example, a pathogen antigen, or a self-antigen, e.g. a tumor-associated antigen. The cloaked RNA may be formulated for delivery to a cell in vivo, e.g. as a vaccine, as gene therapy, for cellular reprogramming, and the like. In such a formulation the RNA may be coated or complexed with a lipid nanoparticle, liposome, lipoplex, etc.

The RNA may be a mixture of different types of RNA and may be in single- or double-stranded form. The RNA may be synthetic or a natural product. An mRNA may or may not have a cap and/or poly A tail. An RNA may or may not contain unnatural modified nucleobases. An RNA may be at least 12 nt in length, at least about 15, at least about 20, at least about 25, and may be greater than about 100 nt, 500 nt, 750 nt, 1 kb, 1.5 kb, 2 kb, or larger. In some embodiments the RNA is greater than about 500 nt, greater than about 750 nt, greater than about 1 Kb in length. An RNA acylated by the methods disclosed herein may comprise at least about 10% acylated 2′-OH, at least about 20% acylated 2′-OH, at least about 30% acylated 2′-OH, at least about 50% acylated 2′-OH, or more, up to substantially the entire RNA. In a population of RNA, there may be at least about 20% acylated 2′-OH, at least about 30% acylated 2′-OH, at least about 50% acylated 2′-OH, or more, up to substantially the entire population. Alternatively, folded RNAs acylated by the methods disclosed herein may comprise at least about 10% acylated 2′-OH, at least about 20% acylated 2′-OH, at least about 30% acylated 2′-OH, at least about 50% acylated 2′-OH, or more of the nucleotides in unpaired loops and regions.

In some embodiments an RNA formulation for therapeutic in vivo delivery to a cell is provided, where the RNA is cloaked by 2′-acylation of at least a portion of the 2′-hydroxyl groups. The RNA in the formulation can be complexed with a carrier, e.g. a lipid nanoparticle, liposome, lipoplex, etc. The formulation may be provided in a pharmaceutically acceptable excipient. The formulation may be provided in a unit dose, e.g. from about 1 mg to about 500 mg of RNA, from about 1 mg, about 5 mg, about 10 mg, about 25 mg, about 50 mg, about 100 mg, about 250 mg, about 500 mg.

Cloaking reagents used for selective 2′ cloaking of RNA include agents with a general structure:

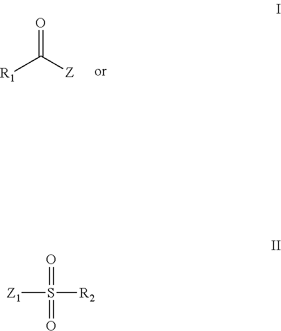

Z is selected from imidazole; 1,2,3-triazole, 1,2,4-triazole, azide, cyanide, anhydride, fluoride, NHS ester; and the like. In some embodiments Z is imidazole.

Z₁ may be selected from imidazole, and a triazole, e.g. 1,2,3-triazole, or 1,2,4-triazole.

R₁ or R₂, which may be generically referred to herein as an “R” group, is a substituted or unsubstituted alkyl group, a substituted or unsubstituted heteroalkyl group, a substituted or unsubstituted aryl or heteroaryl, a substituted or unsubstituted cycloalkyl. In some embodiments R comprises from 1-10 carbons, and optionally comprises 1-4 heteroatoms, particularly N or O. A suitable acyl group is water-soluble, the ester product of which is relatively water-stable, while being electrophilic enough that allows readily reversal by nucleophilic organocatalysts.

Suitable R groups include, for example:

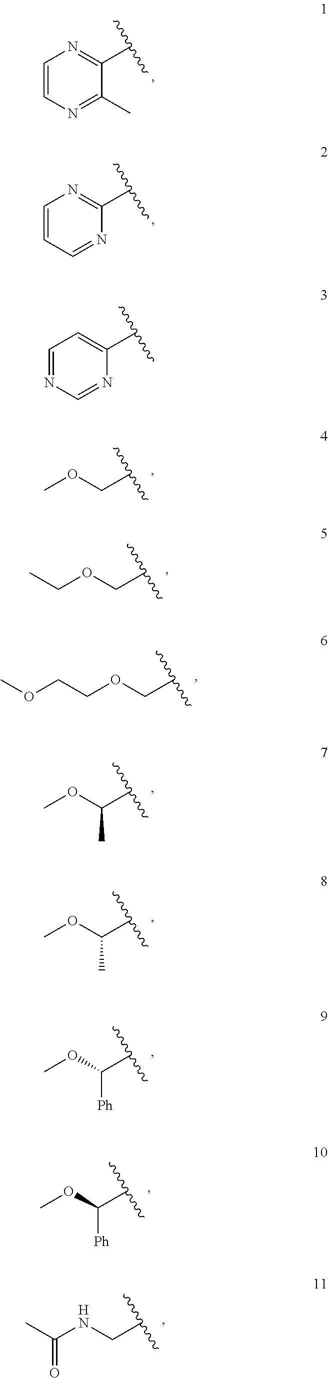

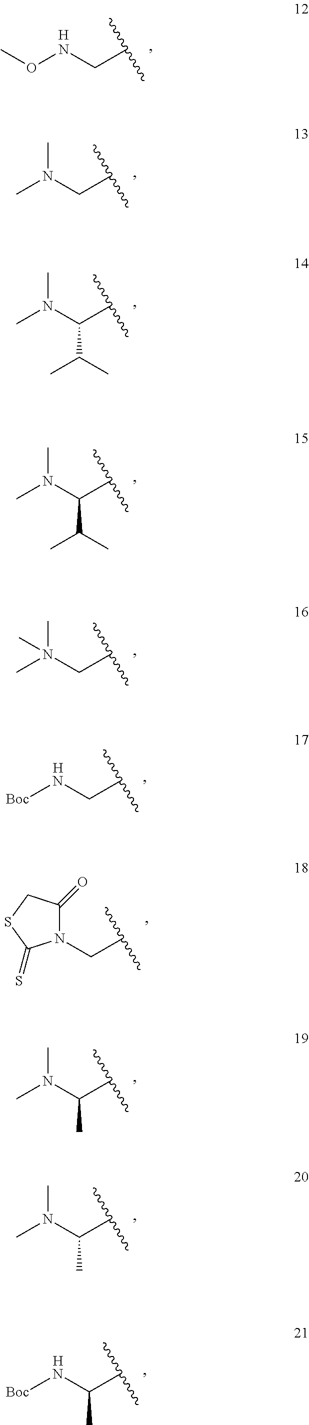

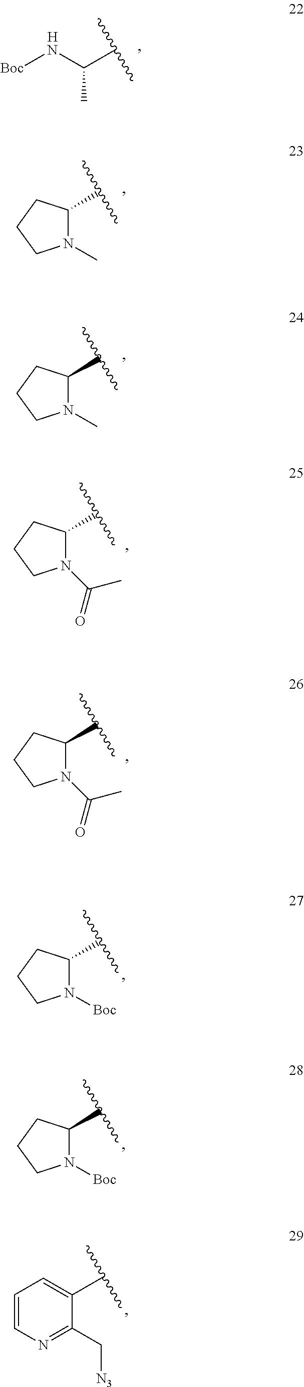

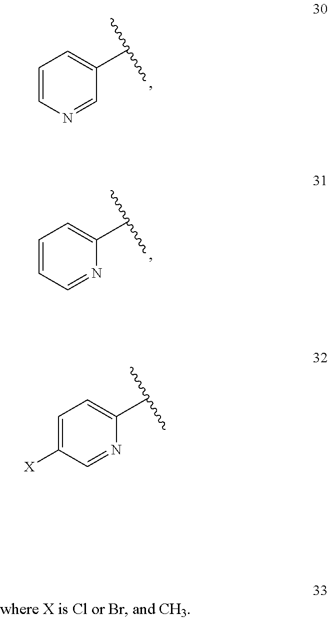

It is shown herein that selection of an R group will influence the effect of the cloaked RNA on cellular RNA sensors, and specific patterns of innate immune mediator expression are associated with exposure to different R groups. For example, exemplified R groups 1, 4, 11, 13, and 19 are shown to provide for distinct expression profiles of immune response mediators including type I interferon, CCL5, IL-8, IL-3, VEGF, for example as shown in FIGS. 9 and 10. In some embodiments an RNA is cloaked with a selected R group to achieve a desired pattern of expression of innate immune system mediators in a cell contacted with the RNA. For example, an R group may be selected in order to reduce undesirable proinflammatory responses.

In some embodiments a method is providing for tuning the innate immune response of a cell with an exogenous RNA composition, the method comprising: cloaking the RNA by 2′-hydroxyl acylation with 1 or more, usually 2 or more, 3 or more, 4 or more different RNA cloaking reagents, for example using a reagent comprising an R group selected from R1-R33 as disclosed herein; contacting a cell, e.g. a relevant human cell, with the cloaked RNA; and measuring the release of innate immune mediators by the cell The level of acylation may be varied, e.g. from about 5% to about 95%. The relevant cell may be a cell type targeted by a therapeutic RNA agent, e.g. an antigen-presenting cell, a cancer cell, a blood cell, etc. The innate immune mediators are optionally selected from TNFα, IL-1β, IFNα, IFNβ, IL-6, CCL5, CXCL8, CXCL10, IL-8, IL-3, VEGF and PDGFAA. The R group providing the desired pattern of innate immune response can be selected for use in cloaking the RNA, e.g. for a therapeutic formulation.

In some embodiments, R is

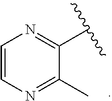 [SMILES: *c1nccnc1C]

In some embodiments, R is

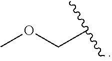 [SMILES: *COC]

In some embodiments R is

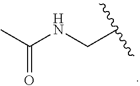 [SMILES: *CNC(C)=O]

In some embodiments R is

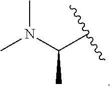 [SMILES: *[C@@H](C)N(C)C]

In some embodiments R is

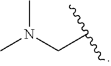 [SMILES: *CN(C)C]

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. N,N-dimethylglycine acylimidazole (DMG-Im, R group 13) acylates RNA 2′-OH groups in high yields.

FIGS. 2A-2C. Luminex human 48-plex cytokine analysis showing cloaking by DMG-Im potently suppressed the expression of some proinflammatory cytokines and immune activation markers.

FIGS. 3A-3B. Luminex human 48-plex cytokine analysis showing cloaking by DMG-Im potently modulates the expression of some proinflammatory cytokines and immune activation markers over time.

FIGS. 4A-4E. Cloaking by DMG-Im differentially modulates certain innate immune responsive pathways over time. (A) VEGF, (B) CCL5, (C) IL6, (D) IL-8) (E) IL4.

FIG. 5. Cloaking with DMG-Im suppressed expression of ISG15.

FIG. 6A-6B. Spontaneous restoration of mRNAs encoding eGFP-mRNA in HeLa, HEK293 and SW480 cells (a) and destabilized GFP (b) that were cloaked with DMG-Im.

FIG. 7. Chemical structures of R groups for immunomodulatory acylimidazole reagents.

FIG. 8. Spontaneous restoration of d2GFP-mRNA translation of selected acylimazole reagents in HEK293 cells

FIGS. 9A-9D. Luminex human 48-plex cytokine analysis showing eGFP-mRNA aliquots that were cloaked by acylimidazoles with diverse chemical structures differentially modulates the expression of some proinflammatory cytokines and immune activation markers.

FIGS. 10A-10C. Luminex human 48-plex cytokine analysis showing chemical structure-dependent reduction in proinflammatory immune responses, comparing DMG to R groups 1, 4, 11 and 19 for expression of IL-8, CCL5 and VEGF.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C. may be contacted for more details). Chemicals that are known but not commercially available in catalogs may be prepared by custom chemical synthesis houses, where many of the standard chemical supply houses (e.g., those listed above) provide custom synthesis services.

The term “alkyl” refers to a C₁-C₂₀ alkyl that may be linear, branched, or cyclic. “Lower alkyl”, as in “lower alkyl”, or “substituted lower alkyl”, means a C—C₁₀ alkyl. The term “alkyl”, “lower alkyl” or “cycloalkyl” includes methyl, ethyl, isopropyl, propyl, butyl, isobutyl, sec-butyl, tert-butyl, pentyl, neopentyl, hexyl, cyclopropyl, cyclopropylmethyl, cyclobutyl, cyclobutylmethyl, cyclopentyl, cyclopentylmethyl, cyclohexyl, cyclohexylmethyl, C₆ to C₁₂ spirocycles, cyclopropylethyl, cyclobutylethyl, decalinyl, Bicyclo-[1.1.1]-pentyl, norboranyl, bicylo-[2.2.2]-octyl, cubyl, adamantanyl and related cage hydrocarbon moieties. In certain embodiments, the alkyl is a C₁-C₂₀ alkyl. In certain embodiments the alkyl group is poly deuterated.

A “substituted alkyl” is an alkyl which is typically mono-, di-, or tri-substituted with heterocycloalkyl, aryl, substituted aryl, heteroaryl, nitro, cyano (also referred to herein as nitrile), azido, halo, —OR, —SR, —SF₅, —CHO, —COR, —C(O)OR, —C(O)—NR₂, —OC(O)R, —OC(O)NR₂, —OC(O)OR, —P(O)(OR)₂, —OP(O)(OR)₂, —NR₂, —N⁺R₃ (wherein a counterion may be present), —CONR₂, —NRCOR, —NHC(O)OR, —NHC(O)NR₂, —NHC(NH)NR₂, SO₃, —SO₂OR, —OSO₂R, —SO₂NR₂, or —NRSO₂R, where each R is, independently, hydrogen, lower alkyl, R′-substituted lower alkyl, aryl, R′-substituted aryl, heteroaryl, heteroaryl(alkyl), R′-substituted aryl(alkyl), or aryl(alkyl) and each R′ is, independently, hydroxy, halo, alkyloxy, cyano, thio, SF₅, nitro, alkyl, halo-alkyl, or amino. Substituted alkyls which are substituted with one to three of the substituents selected from the group consisting of alkynyl, cyano, halo, alkyloxy, thio, nitro, amino, or hydroxy are particularly of interest.

The term “aryl” refers to an aromatic ring having (4n+2) pi electrons that may contain 6 to 20 ring carbon atoms, and be composed of a single ring (e.g., phenyl), or two or more condensed rings, such as 2 to 3 condensed rings (e.g., naphthyl), or two or more aromatic rings, such as 2 to 3 aromatic rings, which are linked by a single bond (e.g., biphenylyl). In certain cases, the aryl is C₆-C₁₆ or C₆ to C₁₄. In certain embodiments the alkyl group has one or more hydrogen atoms replaced with deuterium.

Heteroaryl means an aromatic ring system containing (4n+2)pi electrons and comprised of 1 to 10 ring carbon atoms and 1 to 5 heteroatoms selected from O, N, S, Se, having a single ring (e.g., thiophene, pyridine, pyrazine, imidazole, oxazole, tetrazole, etc.), or two or more condensed rings, for example 2 to 3 condensed rings (e.g., indole, benzimidazole, quinolone, quinoxaline, phenothiazine, etc.), or two or more aromatic rings, such as 2 to 3 aromatic rings, which are linked by a single bond (e.g., bipyridyl). In some cases, the heteroaryl is C₁-C₁₆, and a selection of 1 to 5 heteroatoms consisting of S, Se, N, and 0.

The term “heterocycloalkyl”, “heterocycle”, “heterocyclic group” or “heterocyclyl” refers to a saturated or unsaturated nonaromatic ring system containing 1 to 10 ring carbon atoms and 1 to 5 heteroatoms selected from O, N, S, Se, having a single ring (e.g., tetrahydrofuran, aziridine, azetidine, pyrrolidine, piperidine, tetrathiopyran, hexamethylene oxide, oxazepane, etc.), or two or more condensed rings, such as 2 to 3 condensed rings (e.g., indoline, tetrahydrobenzodiazapines, etc., including fused, bridged and spiro ring systems, having 3-15 ring atoms, included 1 to 4 heteroatoms. In certain cases, the heterocycloalky is C₁-C₁₆, and a selection of 1 to 5 heteroatoms consisting of S, Se, N, and O. In fused ring systems, one or more of the rings can be cycloalkyl, heterocycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, benzimidazole, pyrazole, benzopyrazole, tetrazole, 1,2,3-triazole, benzotriazole, 1,2,4-triazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, benzoisothiazole, phenazine, isoxazole, benzoisooxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, benzothiazole, thiazolidine, furan, benzofuran, thiophene, benzothiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, benzotetrahydrofuranyl, and the like.

Substituted heterocycloalkyl, aryl, heteroaryl are optionally substituted with, hydrogen, 1 to 3 alkyl, alkenyl, alkynyl, cycloalkyl, cycloalkyl(alkyl), aryl, substituted aryl, aryl(alkyl), —SO₂NR⁵R⁵, —PO₃H₂, —NR⁵SO₂R⁶ or —NR⁵C(═O)R⁶, wherein R⁵ and R⁶ are independently, hydrogen, alkyl, alkenyl, alkynyl, cycloalkyl, cycloalkyl(alkyl), aryl, optionally substituted heterocycloalkyl, aryloxy, heteroaryl, heteroaryl(alkyl), or R⁵ and R⁶ together are —(CH₂)₃₋₆— or —(CH₂)₀₋₃X(CH₂)₀₋₃— where X═NR, O, S, SO₂, substituted aryl(alkyl), halo(alkyl), SF₅, NR⁶³, azido, cyano (also referred to herein as nitrile), —OR⁵, —SR⁵, —NR⁵R⁶, halogen, nitro, SCH₃, OCF₃, SO₂CH₃, SCF₃, SO₂CF₃, CF₃, —SO₂OR⁵, —OSO₂R⁵, CCl₃, —C(═O)R⁵, —C(═O)OR⁵; —C(═O)NR⁵R⁶, —OC(═O)R⁵.

By “substituted” as in “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C6 alkenyl), C2-C24 alkynyl (including C2-C18 alkynyl, further including C2-C12 alkynyl, and further including C2-C6 alkynyl), C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

The term “water-soluble group” refers to a functional group that is well solvated in aqueous environments and that imparts improved water solubility to the compound to which it is attached. Water-soluble groups of interest include, but are not limited to, polyalcohols, straight chain or cyclic saccharides, primary, secondary, tertiary, or quaternary amines and polyamines, sulfate groups, sulfonate groups, sulfinate groups, carboxylate groups, phosphate groups, phosphonate groups, phosphinate groups, ascorbate groups, glycols, including polyethylene glycols (PEG) and modified PEGs, and polyethers. In some instances, water-soluble groups are primary, secondary, tertiary, and quaternary amines, carboxylates, phosphonates, phosphates, sulfonates, sulfates, —N(H)₀₋₁(CH₂CH₂OH)₁₋₂, —NHCH₂CH₂N(CH₃)₂₋₃, —NHCH₂CH₂SO₃H, —NHCH₂CH₂PO₃H₂ and —NHCH₂CH₂CO₂H, —(CH₂CH₂O)_{yy}CH₂CH₂XR^{yy}, —(CH₂CH₂O)_{yy}CH₂CH₂X—, —X(CH₂CH₂O)_{yy}CH₂CH₂—, glycol, oligoethylene glycol, and polyethylene glycol, wherein yy is selected from 1 to 1000, X is selected from O, S, and NR^{ZZ}, and R^{ZZ} and R^{YY} are independently selected from H and C1-3 alkyl.

The term “carboxy isostere” refers to standard medicinal bioisosteric replacement groups for carboxylic acids, amides and ester. These include, but are not limited to: acyl cyanamide, tetrazoles, hydroxychromes, 3-hydroxy-1,2,4-triazoles, 1-hydroxy pyrazoles, 2,4-dihydroxy imidazoles, 1-hydroxy imidazole, 1-hydroxy 1,2,3-triazole, alkylsulfonyl carboxamides, hydroxy isoxazoles, 5-hydroxy 1,2,4-oxadiazoles, thiazoles, 1,2,4-oxadiazoles, 1,2,4-oxadiazolones, oxazoles, triazoles, thiazoles, others hydroxamic acids, sulfonimide, acylsulfonamide, sulfonylureas, oxadiazolone, thiazolidinediones, oxadiazole, thiadiazole, isothiazoles, difluorophenols, tetramic acids, tetronic acids, squaric acids, hydroxyquinoline-ones, hydroxyquinoline-2-ones, boronic acids and phosphoric acids.

As used herein the term “PEG” refers to a polyethylene glycol or a modified polyethylene glycol. Modified polyethylene glycol polymers include a methoxypolyethylene glycol, and polymers that are unsubstituted or substituted at one end with an alkyl, a substituted alkyl or a substituent (e.g., as described herein).

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH₂), mono-substituted C1-C24 alkylcarbamoyl (—(CO)—NH(C1-C24 alkyl)), di-substituted alkylcarbamoyl (—(CO)—N(C1-C24 alkyl)₂), mono-substituted arylcarbamoyl (—(CO)—NH-aryl), thiocarbamoyl (—(CS)—NH₂), carbamido (—NH—(CO)—NH₂), cyano (—C═N), isocyano (—N+≡C—), cyanato (—O—C═N), isocyanato (—O—N+≡C—), isothiocyanato (—S—C═N), azido (—N═N+=N—), formyl (—(CO)—H), thioformyl (—(CS)—H), amino (—NH₂), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C1-C24 alkylsulfanyl (—S— alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO₂-alkyl), C5-C20 arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

When the term “substituted” appears prior to a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O)₂ (M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O) O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸OR⁸O, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as +N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl, N-morpholinyl, —N(H)₀₋₁(CH₂CH₂OH)₁_2, —NHCH₂CH₂N(CH₃)₂₋₃, —NHCH₂CH₂SO₃H, —NHCH₂CH₂PO₃H₂and —NHCH₂CH₂CO₂H.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸OR⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸OR⁸⁰, —OC(O)R⁷⁰, —OC(S)R⁷⁰, —OCO₂⁻, M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O—)₂(M⁺)₂, —P(O)(OR⁷⁰O⁻M⁺, —P(O)(OR⁷⁰)(OR⁷⁰), —C(O)R⁷⁰, —C(S) R⁷⁰, —C(NR⁷⁰)R⁷⁰, —C(O)OR⁷⁰, —C(S)OR⁷⁰, —C(O)NR⁸OR⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺are as previously defined.

Salts include but are not limited to: Na, K, Ca, Mg, ammonium, tetraalkyl ammonium, aryl and alkyl sulfonates, phosphates, carboxylates, sulfates, Cl, Br, and guanidinium.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “heterocycloalkyl(alkyl)” refers to the group (heterocycloalkyl)-(alkyl)-.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. Polymorphic, pseudo-polymorphic, amorphous and co-crystal forms of a compound are also of interest. All such forms are embraced by the present disclosure. Thus, the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

Innate Immune Responses. The presence and localization of infectious microorganisms are detected in mammalian cells by pattern recognition receptors (PRRs). These receptors are ligand-specific sensors that are able to recognize both pathogen-associated molecular patterns (PAMPs) and damage-associated molecular patterns (DAMPs) to orchestrate an early host defense against an infection or injury. Exogenous nucleic acids are one of the PAMP classes, and their molecular features, such as length, double- or single-strand configuration, modification of nucleosides, and sequence motifs, play a key role in immune recognition.

DAMPs are endogenous host-derived danger signals that are released by damaged or dying cells or upon cellular stress to the extracellular or intracellular space, promoting inflammation in order to clean the tissue from debris for subsequent regeneration. The most studied DAMPs include mono- and polysaccharides (glycans), high-mobility group box 1 (HMGB1), nucleic acids, and secreted ATP.

There are several types of PRRs that sense nucleic acids in mammalian cells, which are located in different cell compartments-on the plasma membrane, in endosomes, and in the cytoplasm-which allows sensors to detect both PAMP and DAMP nucleic acids. The first one is the endosomal subfamily of TLRs (TLR3, 7, 8, and 9). Another type includes cytosolic RNA-binding proteins such as retinoic acid-inducible gene I (RIG-1), melanoma differentiation-associated protein 5 (MDA5), and laboratory of genetics and physiology protein 2 (LGP2). Another type of recently described sensor is cytoplasmic DNA sensors, which are DNA-dependent activators of IRFs (DAI).

Each endosomal TLR is able to recognize a specific type of nucleic acid: dsRNA activates TLR3, non-self ssRNA triggers TLR 7 and TLR8, and CpG DNA triggers TLR9. TLR3 is expressed in myeloid dendritic cells; therefore, it connects the innate and adaptive immune systems, and the other endosomal TLRs are expressed in a wider variety of immune cells, including pDCs, macrophages, monocytes, and lymphocytes.

Cytosolic RNA-binding proteins or RIG-1 like receptors (RLRs) include three members: RIG-1, MDA5, and LGP2. RLRs belong to the SF2 helicase super-family, which are mostly found in the cytoplasm, but some amount of RIG-1 is located in the nucleus. RIG-1 and MDA5 are signaling proteins, while LGP2 has a regulatory role. These different functions are due to their structural dissimilarities. RIG-1 and MDA5 have a similar structure, with a helicase domain in the middle part and a carboxy-terminal domain (CTD). Both of these domains are able to detect and bind RNA; moreover, they both possess caspase activation and recruitment domains (CARDs), which mediate signal transduction and lead to type I IFN gene expression. Although these proteins share structural similarities and a downstream conserved signaling pathway, they are activated by distinct RNA species. RIG-1 prefers binding with short dsRNA, which is tri-phosphorylated at the 5′ end. Moreover, RIG-1 can distinguish between 5′-diphosphate and 5′-triphosphate dsRNA. These energetic differences of binding with mono-, di-, or triphosphate enables RIG-1 to discriminate between endogenous and viral RNA. On the contrary, MDA5 is activated by long dsRNA, which was confirmed by its activation by poly(I:C), a synthetic mimic of long dsRNA.

The binding of ligands to TLR stimulates specific intracellular downstream signaling cascades that initiate host defense reactions, leading to production of pro-inflammatory cytokines and type 1 interferon. TLR signaling depends on the nature of the stimulus, the activated TLR, and the downstream adaptor molecule. Different types of signaling adaptor proteins can be recruited by the TIR domain, of which myeloid differentiation primary-response protein 88 (MyD88) is essential for TLR2, 4, 5, 7, 8, and 9, leading to the production of inflammatory cytokines. TIR domain-containing adaptor protein inducing IFN-p (TRIF). TRIF acts independently of MyD88 in signal transduction following TLR3 and 4 activation and results in production of type 1 interferon. Released innate immune mediators, which may be proinflammatory agents, include without limitation, TNFα, IL-1β, IFNα, IFNβ, IL-6, CCL5, CXCL8, CXCL10, IL-8, IL-3, VEGF and PDGFAA.

### Methods of RNA Cloaking

RNA is cloaked to reduce triggering of innate immune responses by (i) contacting, in aqueous solution, RNA with a cloaking reagent; and (ii) reacting the RNA with the reagent to produce modified RNA comprising acylated 2′-OH ribose. The acylation may be reversed spontaneously when the RNA is inside a cell.

Cloaking reagents useful in the methods disclosed herein may have the general structure:

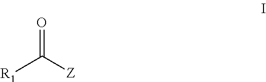

where R₁ is a substituted or unsubstituted alkyl group, a substituted or unsubstituted heteroalkyl group, a substituted or unsubstituted aryl or heteroaryl, a substituted or unsubstituted cycloalkyl. In some embodiments R comprises from 1-10 carbons, and optionally comprises 1-4 heteroatoms, particularly N or 0.

Z is selected from imidazole; 1,2,3-triazole, 1,2,4-triazole, azide, cyanide, anhydride, fluoride, NHS ester; and the like. In some embodiments Z is imidazole.

In other embodiments a cloaking reagent has a general structure:

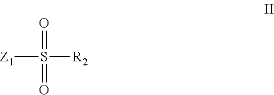

Z₁ may be selected from imidazole, and a triazole, e.g. 1,2,3-triazole, or 1,2,4-triazole.

Suitable R₁ or R₂ groups (collectively an R group) for formula I or II include, for example:

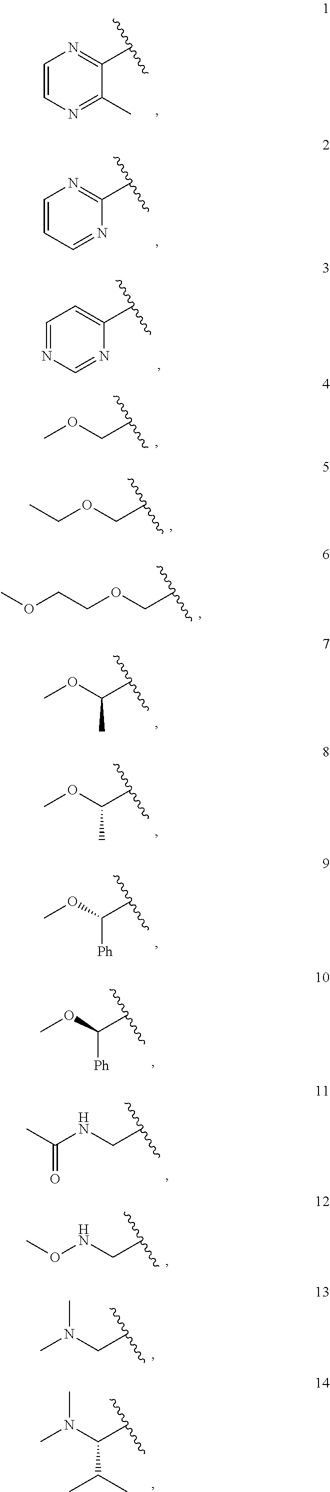

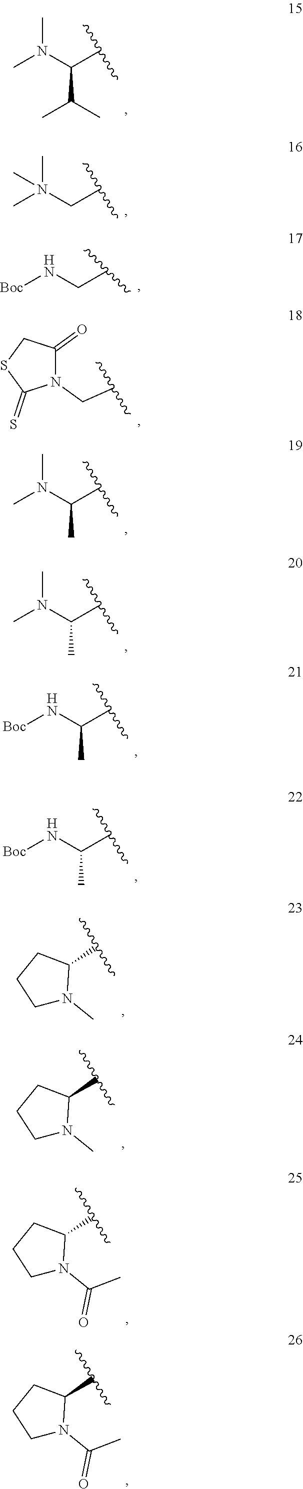

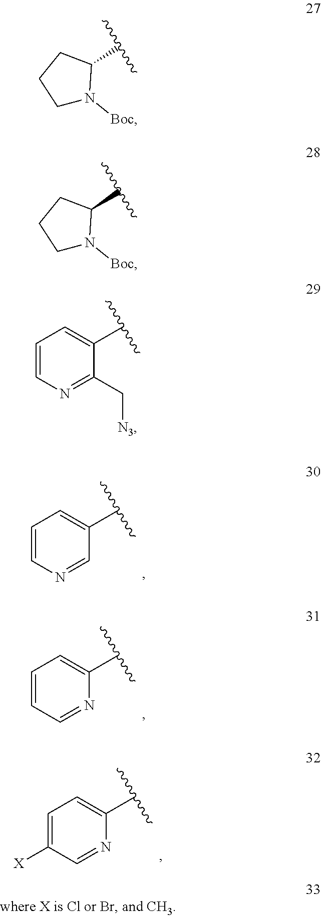

In some embodiments a targeted region of the RNA is cloaked, where a region of the RNA, e.g. one or more of the 5′-UTR, all or a portion of the open reading frame, 3′-UTR of the mRNA is hybridized with complementary DNA oligos, with a length ranged from about 18 nt to about 120 nt. The non-hybridized region of the mRNA-DNA hybrids are then selectively modified with cloaking reagent of Formula I or II. Subsequent removal of complementary DNA oligos with DNases produces mRNA with selective 2′-modifications. In some embodiments selective acylation is achieved with largely unfolded RNAs, e.g. RNA in water without added cations, or at elevated temperature, or with added denaturants. In some embodiments modification is targeted primarily to unpaired regions of otherwise folded RNAs.

Bioorthogonal methods are provided for optional reversal of 2′-OH RNA acylation with water-soluble organocatalysts that are a strong nucleophile and weak base, performed in aqueous solution at neutral pH, e.g. at a pH from about 7 to about 8, including pH 7.1, 7.2, 7.3, 7.4, 7.5, 7.6, 7.7, 7.8, 7.9, etc. In some embodiments the organocatalyst is Tris (tris(hydroxymethyl)aminomethane). In some embodiments the organocatalyst is DABCO (1,4-diazabicyclo[2.2.2]octane).

Buffers for reversal of acylation include, without limitation Tris (tris(hydroxymethyl)aminomethane), DABCO (1,4-diazabicyclo[2.2.2]octane), NaCN, etc. Buffers may be present at a concentration of from about 1 mM, about 5 mM, about 10 mM, about 25 mM, about 50 mM, about 100 mM, and not more than about 250 mM. The reaction is performed at a temperature from room temperature to 37° C., for a period of from about 1 minute to about 24 hours, from about 30 minutes to about 12 hours.

Upon reversal of acylation, less than about 75% of the RNA may comprise acylated 2′-OH, less than about 50%, less than about 25%. The de-acylated RNA is biologically active, and can be used in hybridization, translation, reverse transcription, Cas9-mediated gene editing, etc. reactions. In other embodiments the cloaking is not reversed, for example where the acylation is present outside of the coding region, or at other regions of the sequence where the acylation does not substantially reduce translation.

It is also shown that there can be a spontaneous reversal of acylation in an intracellular environment, e.g. after the cloaked mRNA is delivered to a cell for expression.

### Therapeutic Formulations

Compositions comprising RNA cloaked by acylated 2′-OH ribose are provided, where the RNA modification is performed according to the methods disclosed herein. In some embodiments the composition is formulated with a pharmaceutically acceptable excipient. In some embodiments the acylated RNA is formulated for delivery to a mammalian cell, e.g. as a vaccine, gene therapy, delivery of biologically active anti-sense oligonucleotides, delivery of sequences encoding a therapeutic protein, delivery of reprogramming factors, and the like.

The therapeutic modified RNA may be mRNA, anti-sense mRNA, RNAi, synthetic RNA such as chemically synthesized or in vitro transcribed forms, or any other form of RNA. In some specific embodiments the RNA is mRNA. An RNA population acylated by the methods disclosed herein may comprise at least about 10% acylated 2′-OH, at least about 20% acylated 2′-OH, at least about 30% acylated 2′-OH, at least about 50% acylated 2′-OH, or more. Where the modification is selectively present in the poly(A)-tail, the RNA 5′-UTR, open reading frame, 3′-UTR of mRNA may be substantially free of acylated 2′OH, while the poly(A) tail may comprise at least about 30% acylated 2′OH, at least about 50% acylated 2′-OH, at least about 75% acylated 2′-OH, at least about 90% acylated 2′-OH, or more. In some embodiments the R group and level of acylation is selected to provide a desired profile of innate immune responses.

In some embodiments a therapeutic formulation comprises an RNA formulated with a carrier, where the term “carrier” denotes an organic or inorganic ingredient, natural or synthetic, with which the RNA is combined to facilitate administration. In some embodiments, a formulation comprises at least one RNA (e.g., mRNA) polynucleotide species having an open reading frame encoding an antigen. In some embodiments the carrier is a lipid nanoparticle (LNP), a polymeric nanoparticle, a lipid carrier such as a lipidoid, a liposome, a lipoplex, a peptide carrier, a nanoparticle mimic, a nanotube, or a conjugate.

In some embodiments an RNA formulation is an RNA vaccine formulation. Where the formulation is a vaccine, the vaccine may be a cancer vaccine, pathogen vaccine, etc. A cancer vaccine, for instance, is a vaccine including a cancer antigen that is known to be found in cancers or tumors generally or in a specific type of cancer or tumor. Antigens that are expressed in or by tumor cells are referred to as “tumor associated antigens”. A particular tumor associated antigen may or may not also be expressed in non-cancerous cells. Many tumor mutations are known in the art. Personalized cancer vaccines may include RNA encoding for one or more known cancer antigens specific for the tumor or cancer antigens specific for each subject, referred to as neoepitopes or patient specific epitopes or antigens. A “patient specific cancer antigen” is an antigen that has been identified as being expressed in a tumor of a particular patient. The patient specific cancer antigen may or may not be typically present in tumor samples generally. Tumor associated antigens that are not expressed or rarely expressed in non-cancerous cells, or whose expression in non-cancerous cells is sufficiently reduced in comparison to that in cancerous cells and that induce an immune response induced upon vaccination, are referred to as neoepitopes.

Vaccine formulations may comprise, for example, an mRNA encoding an antigen of interest. mRNA vaccines may comprise one or more antigens. In some embodiments an mRNA vaccine comprises 3 or more, 4 or more, 5 or more, 6 or more, 7 or more, 8 or more, 9, or more antigens. In one embodiment, the antigen is derived from a human pathogen. In other embodiments, the antigen is a tumor-associated antigen, e.g. a cancer neoantigen.

In some embodiments the mRNA comprises at least one chemically modified nucleobase, sugar, backbone, or any combination thereof in addition to the acylated 2′-OH ribose. In embodiments the at least one chemically modified nucleobase is selected from the group consisting of pseudouracil (ψ), N1-methylpseudouracil (m1ψ), 1-ethylpseudouracil, 2-thiouracil (s2U), 4′-thiouracil, 5-methylcytosine, 5-methyluracil, 5-methoxyuracil, N6-methyladenine, and any combination thereof.

The invention also encompasses infectious disease vaccines, where the mRNA encodes a viral or bacterial antigen. In some embodiments the infectious agent is a strain of virus selected from the group consisting of coronavirus, e.g. SARS, CARS-CoV2, etc., adenovirus; Herpes simplex, type 1; Herpes simplex, type 2; encephalitis virus, papillomavirus, Varicella-zoster virus; Epstein-barr virus; Human cytomegalovirus; Human herpes virus, type 8; Human papillomavirus; BK virus; JC virus; Smallpox; polio virus; Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; Yellow Fever virus; Dengue virus; West Nile virus; Rubella virus; Hepatitis E virus; Human Immunodeficiency virus (HIV); Influenza virus; Guanarito virus; Junin virus; Lassa virus; Machupo virus; Sabid virus; Crimean-Congo hemorrhagic fever virus; Ebola virus; Marburg virus; Measles virus; Mumps virus; Parainfluenza virus; Respiratory syncytial virus; Human metapneumovirus; Hendra virus; Nipah virus; Rabies virus; Hepatitis D; Rotavirus; Orbivirus; Coltivirus; Banna virus; Human Enterovirus; Hanta virus; West Nile virus; Middle East Respiratory Syndrome Corona Virus; Japanese encephalitis virus; Vesicular exanthernavirus; and Eastern equine encephalitis.

In other embodiments, the virus is a strain of Influenza A or Influenza B or combinations thereof. In some embodiments, the strain of Influenza A or Influenza B is associated with birds, pigs, horses, dogs, humans or non-human primates. In some embodiments, the antigenic polypeptide encodes a hemagglutinin protein or fragment thereof. In some embodiments, the hemagglutinin protein is H1, H2, H3, H4, H5, H6, H7, H8, H9, H10, H11, H12, H13, H14, H15, H16, H17, H18, or a fragment thereof. In some embodiments, the hemagglutinin protein does not comprise a head domain (HA1). In some embodiments, the hemagglutinin protein comprises a portion of the head domain (HA1). In some embodiments, the virus is selected from the group consisting of H1N1, H3N2, H7N9, and H10N8.

In some embodiments, the infectious agent is a strain of bacteria selected from Tuberculosis (Mycobacterium tuberculosis), clindamycin-resistant Clostridium difficile, fluoroquinolone-resistant Clostridium difficile, methicillin-resistant Staphylococcus aureus (MRSA), multidrug-resistant Enterococcus faecalis, multidrug-resistant Enterococcus faecium, multidrug-resistance Pseudomonas aeruginosa, multidrug-resistant Acinetobacter baumannii, and vancomycin-resistant Staphylococcus aureus (VRSA). In some embodiments, the bacterium is Clostridium difficile.

In some embodiments the RNA, e.g. an RNA vaccine, is formulated in a lipid nanoparticle (LNP). The use of LNPs enables the effective delivery of RNA. In one embodiment, a lipid nanoparticle comprises lipids including an ionizable lipid (such as an ionizable cationic lipid), a structural lipid, a phospholipid, and acylated RNA. Each of the LNPs described herein may be used as a formulation for the RNA described herein. In one embodiment, a lipid nanoparticle comprises an ionizable lipid, a structural lipid, a phospholipid, and mRNA. In some embodiments, the LNP comprises an ionizable lipid, a PEG-modified lipid, a phospholipid and a structural lipid. In some embodiments, the LNP has a molar ratio of about 20-60% ionizable lipid: about 5-25% phospholipid: about 25-55% structural lipid; and about 0.5-15% PEG-modified lipid. In some embodiments, the LNP comprises a molar ratio of about 50% ionizable lipid, about 1.5% PEG-modified lipid, about 38.5% structural lipid and about 10% phospholipid. In some embodiments, the LNP comprises a molar ratio of about 55% ionizable lipid, about 2.5% PEG lipid, about 32.5% structural lipid and about 10% phospholipid. In some embodiments, the ionizable lipid is an ionizable amino or cationic lipid and the phospholipid is a neutral lipid, and the structural lipid is a cholesterol. In some embodiments, the LNP has a molar ratio of 50:38.5:10:1.5 of ionizable lipid: cholesterol:DSPC: PEG2000-DMG.

Ionizable lipids can be selected from the non-limiting group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)[1,3]-dioxolane (DLin-KC2-DMA), 1,2-dioleyloxy-N,N-dimethylaminopropane (DODMA), (13Z,165Z)-N,N-dimethyl-3-nonydocosa-13-16-dien-i-amine (L608), 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-{8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3s)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

The lipid composition of the pharmaceutical composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In certain embodiments, a phospholipid useful or potentially useful in the present invention is an analog or variant of DSPC. In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. In certain embodiments, an alternative lipid is used in place of a phospholipid of the invention.

The LNPs disclosed herein can comprise one or more structural lipids. As used herein, the term “structural lipid” refers to sterols and also to lipids containing sterol moieties. Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In one embodiment, the amount of the structural lipid (e.g., an sterol such as cholesterol) in the lipid composition of a pharmaceutical composition disclosed herein ranges from about 20 mol % to about 60 mol %, from about 25 mol % to about 55 mol %, from about 30 mol % to about 50 mol %, or from about 35 mol % to about 45 mol %. In one embodiment, the amount of the structural lipid (e.g., an sterol such as cholesterol) in the lipid composition disclosed herein ranges from about 25 mol % to about 30 mol %, from about 30 mol % to about 35 mol %, or from about 35 mol % to about 40 mol %. In one embodiment, the amount of the structural lipid (e.g., a sterol such as cholesterol) in the lipid composition disclosed herein is about 24 mol %, about 29 mol %, about 34 mol %, or about 39 mol %. In some embodiments, the amount of the structural lipid (e.g., an sterol such as cholesterol) in the lipid composition disclosed herein is at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

The lipid composition of a pharmaceutical composition disclosed herein can comprise one or more a polyethylene glycol (PEG) lipid. As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the lipid moiety of the PEG-lipids includes those having lengths of from about C14 to about C22, preferably from about C14 to about C16. In some embodiments, a PEG moiety, for example an mPEG-NH2, has a size of about 1000, 2000, 5000, 10,000, 15,000 or 20,000 daltons. In one embodiment, the PEG-lipid is PEG2k-DMG. In one embodiment, the lipid nanoparticles described herein can comprise a PEG lipid which is a non-diffusible PEG. Non-limiting examples of non-diffusible PEGs include PEG-DSG and PEG-DSPE. PEG-lipids are known in the art, such as those described in U.S. Pat. No. 8,158,601 and International Publ. No. WO 2015/130584 A2, which are incorporated herein by reference in their entirety.

In one embodiment, PEG lipids useful in the present invention can be PEGylated lipids described in International Publication No. WO2012/099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %. In some aspects, the lipid composition of the pharmaceutical compositions disclosed herein does not comprise a PEG-lipid.

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

The ratio between the lipid composition and the RNA range can be from about 10:1 to about 60:1 (wt/wt). In some embodiments, the ratio between the lipid composition and the acylated RNA can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1. In one embodiment, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, RNAs encoding an antigen polypeptide are formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

In addition to LNPs, the acylated RNA may be formulated in other carriers such as liposomes, lipoids and lipoplexes, particulate or polymeric nanoparticles, peptide carriers, nanoparticle mimics, nanotubes, conjugates, or emulsion delivery systems such as cationic submicron oil-in-water emulsions.

Liposomes are amphiphilic lipids which can form bilayers in an aqueous environment to encapsulate a RNA-containing aqueous core. These lipids can have an anionic, cationic or zwitterionic hydrophilic head group. Liposomes can be formed from a single lipid or from a mixture of lipids. A mixture may comprise (i) a mixture of anionic lipids (ii) a mixture of cationic lipids (iii) a mixture of zwitterionic lipids (iv) a mixture of anionic lipids and cationic lipids (v) a mixture of anionic lipids and zwitterionic lipids (vi) a mixture of zwitterionic lipids and cationic lipids or (vii) a mixture of anionic lipids, cationic lipids and zwitterionic lipids. Similarly, a mixture may comprise both saturated and unsaturated lipids. Exemplary phospholipids include, but are not limited to, phosphatidylethanolamines, phosphatidylcholines, phosphatidylserines, and phosphatidylglycerols. Cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are DPPC, DOPC and dodecylphosphocholine. The lipids can be saturated or unsaturated.

Polymeric microparticles or nanoparticles can also be used to encapsulate or adsorb the acylated RNA. The particles may be substantially non-toxic and biodegradable. The particles useful for delivering RNA may have an optimal size and zeta potential. For example, the microparticles may have a diameter in the range of 0.02 μm to 8 μm. When the composition has a population of micro- or nanoparticles with different diameters, at least 80%, 85%, 90%, or 95% of those particles ideally have diameters in the range of 0.03-7 μm. The particles may also have a zeta potential of between 40-100 mV, in order to provide maximal adsorption of the RNA to the particles.

Non-toxic and biodegradable polymers include, but are not limited to, poly(ahydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof. In some embodiments, the particles are formed from poly(ahydroxy acids), such as a poly(lactides) (“PLA”), copolymers of lactide and glycolide such as a poly(D,L-lactide-co-glycolide) (“PLG”), and copolymers of D,L-lactide and caprolactone. Useful PLG polymers include those having a lactide/glycolide molar ratio ranging, for example, from 20:80 to 80:20 e.g. 25:75, 40:60, 45:55, 55:45, 60:40, 75:25. Useful PLG polymers include those having a molecular weight between, for example, 5,000-200,000 Da e.g. between 10,000-100,000, 20,000-70,000, 40,000-50,000 Da.

Oil-in-water emulsions may also be used for delivering the acylated RNA to a subject. Examples of oils useful for making the emulsions include animal (e.g., fish) oil or vegetable oil (e.g. nuts, seeds and grains). The oil may be biodegradable (metabolizable) and biocompatible. Some exemplary oils include tocopherols and squalene, a shark liver oil which is a branched, unsaturated terpenoid and combinations thereof. Terpenoids are branched chain oils that are synthesized biochemically in 5-carbon isoprene units.

The aqueous component of the emulsion can be water or can be water in which additional components have been added. For instance, it may include salts to form a buffer e.g. citrate or phosphate salts, such as sodium salts. Exemplary buffers include: a phosphate buffer; a Tris buffer; a borate buffer; a succinate buffer; a histidine buffer; or a citrate buffer.

The oil-in water emulsions ideally include one or more cationic molecules. For instance, a cationic lipid can be included in the emulsion to provide a positively charged droplet surface to which negatively-charged mRNA can attach. Useful cationic lipids include, but are not limited to: 1,2-dioleoyloxy-3-(trimethylammonio)propane (DOTAP), 3′-[N-(N′,N′-Dimethylaminoethane)-carbamoyl]Cholesterol (DC Cholesterol), dimethyldioctadecyl-ammonium (DDA e.g. the bromide), 1,2-Dimyristoyl-3-Trimethyl-AmmoniumPropane (DMTAP), dipalmitoyl(C16:0)trimethyl ammonium propane (DPTAP), distearoyltrimethylammonium propane (DSTAP). Other useful cationic lipids are: benzalkonium chloride (BAK), benzethonium chloride, cetramide (which contains tetradecyltrimethylammonium bromide and possibly small amounts of dedecyltrimethylammonium bromide and hex adecyltrimethyl ammonium bromide), cetylpyridinium chloride (CPC), cetyl trimethylammonium chloride (CTAC), N,N′,N′-polyoxyethylene (10)-N-tallow-1,3-diaminopropane, dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-naminium chloride (DEBDA), dialkyldimethylammonium salts, [1-(2,3-dioleyloxy)-propyl]-N,N,N,trimethylammonium chloride, 1,2-diacyl-3-(trimethylammonio) propane (acyl group=dimyristoyl, dipalmitoyl, distearoyl, dioleoyl), 1,2-diacyl-3 (dimethylammonio)propane (acyl group=dimyristoyl, dipalmitoyl, distearoyl, dioleoyl), 1,2-dioleoyl-3-(4′-trimethyl-ammonio)butanoyl-sn-glycerol, 1,2-dioleoyl 3-succinyl-sn-glycerol choline ester, cholesteryl (4′-trimethylammonio) butanoate), N-alkyl pyridinium salts (e.g. cetylpyridinium bromide and cetylpyridinium chloride), N-alkylpiperidinium salts, dicationic bolaform electrolytes (C12Me6; C12BU6), dialkylglycetylphosphorylcholine, lysolecithin, L-.alpha.dioleoylphosphatidylethanolamine, cholesterol hemisuccinate choline ester, lipopolyamines, including but not limited to dioctadecylamidoglycylspermine (DOGS), dipalmitoyl phosphatidylethanol-amidospermine (DPPES), lipopoly-L (or D)-lysine (LPLL, LPDL), poly(L (or D)-lysine conjugated to N-glutarylphosphatidylethanolamine, didodecyl glutamate ester with pendant amino group (C GluPhCnN), ditetradecyl glutamate ester with pendant amino group (C14GluCnN+), cationic derivatives of cholesterol, including but not limited to cholesteryl-3β-oxysuccinamidoethylenetrimethylammonium salt, cholesteryl-3β-oxysuccinamidoethylene-dimethylamine, cholesteryl-3β-carboxyamidoethylenetrimethylammonium salt, and cholesteryl-3β-carboxyamidoethylenedimethylamine.

In addition to the oil and cationic lipid, an emulsion can include a non-ionic surfactant and/or a zwitterionic surfactant. Such surfactants include, but are not limited to: the polyoxyethylene sorbitan esters surfactants, especially polysorbate 20 and polysorbate 80; copolymers of ethylene oxide, propylene oxide, and/or butylene oxide, linear block copolymers; octoxynols; (octylphenoxy)polyethoxyethanol; phospholipids such as phosphatidylcholine; polyoxyethylene fatty ethers derived from lauryl, cetyl, stearyl and oleyl alcohols; polyoxyethylene-9-lauryl ether; and sorbitan esters.

Formulations may be provided in a unit dosage form, where the term “unit dosage form,” refers to physically discrete units suitable as unitary dosages for subjects, each unit containing a predetermined quantity of active agent in an amount calculated sufficient to produce the desired effect in association with a pharmaceutically acceptable diluent, carrier or vehicle. The specifications for the unit dosage forms of the present invention depend on the particular RNA and formulation employed and the effect to be achieved, and the pharmacodynamics associated with each formulation in the host. In some embodiments the unit dose is an effective amount for achieving a desired effect, for example, expression of a protein encoded by the modified mRNA, e.g. from about 1 mg to about 500 mg of RNA, from about 1 mg, about 5 mg, about 10 mg, about 25 mg, about 50 mg, about 100 mg, about 250 mg, about 500 mg. Alternatively, depending on use and on the administration route, the modified RNA may be present in a unit dose at a range of from about 100 ng, 1 g, 10 μg, 100 g, 1 mg, up to about 10 mg, up to about 100 mg, up to about 1 g, up to about 10 g, up to about 100 g, etc. Dosages will be appropriately adjusted for the desired use.

The modified RNA and carrier can be formulated with an a pharmaceutically acceptable excipient. A suitable carrier includes sterile saline although other aqueous and non-aqueous isotonic sterile solutions and sterile suspensions known to be pharmaceutically acceptable are known to those of ordinary skill in the art.

The formulation may comprise, depending on the desired use, pharmaceutically-acceptable, non-toxic excipients or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the formulation may also include other carriers, adjuvants, or nontoxic, nontherapeutic, nonimmunogenic stabilizers and the like. In pharmaceutical dosage forms, the modified RNA may be provided in the form of pharmaceutically acceptable salts.

The RNA can be combined with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the active agents can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc. As used herein, the terms “treatment,” “treating,” and the like, refer to administering an agent, or carrying out a procedure, for the purposes of obtaining an effect on or in a subject, individual, or patient. Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician.

### Kits

Kits may be provided. Kits may include reagents suitable for modifying RNA, for example reagents for modification of 2-OH groups of RNA with cloaking reagents as disclosed herein, for example an acylimidazole, sulfonylimidazole, sulfonyltriazole, etc. Components may be separately packaged in two or more containers suitable for use in the methods disclosed herein. Kits may comprise reagents for packaging RNA for in vivo delivery. Kits may also include tubes, buffers, etc., and instructions for use.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Methods and Modifications for Suppressing Innate Immune Responses to RNA

RNA has emerged as a novel modality for therapeutics and vaccination. We have shown that 2′-OH acylation of RNAs (“cloaking”) with reagents disclosed herein can block RNA interactions with cellular proteins and nucleic acids. Although little structural information is available about these receptor-nucleotide complexes, we hypothesized that RNA 2′-modification by acylating reagents might disrupt recognition by RNA receptors and suppress the innate immune responses. Moreover, we hypothesized that the variation of the chemical structures of acylating reagents may fine-tune RNA immunogenicity by differential modulation of innate immune response pathways.

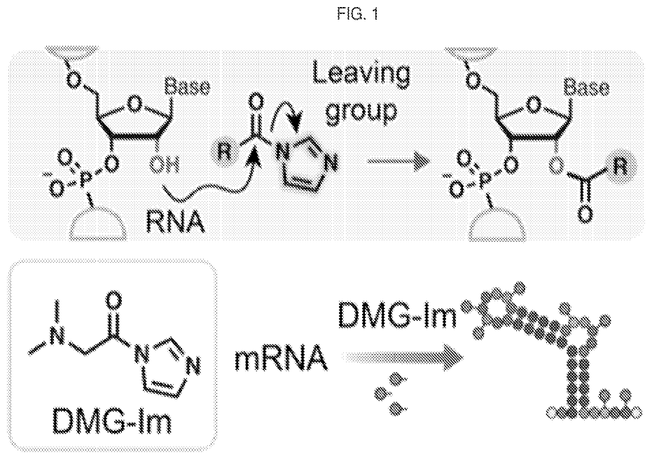

No report has yet shown how 2′-OH acylation affects the immunogenicity of long RNA (>600 nt), in large part because high levels of 2′-modifications (e.g., 2′-F, 2′-OMe) cannot be efficiently incorporated into RNAs with lengths beyond ~150 nt in synthetically accessible yield. To test this, we first prepared a 2′-polyacylated (“cloaked”) model mRNA by reacting a widely used eGFP-mRNA (996 nt) with N,N-dimethylglycine acylimidazole (DMG-Im) (FIG. 1), an acylimidazole reagent that selectively acylates the 2′-hydroxyl groups in high yields. This provided ~50% cloaking in the unpaired accessible regions of eGFP-mRNA. We were attracted to 2′-acylation by acylimidazoles due to their structural simplicity, their ease of production, high solubility in aqueous solutions, and their high-yield acylation of 2′-hydroxyl groups. In addition, acylimidazoles can selectively react with 2′-hydroxyls rather than the nucleobases and can be reversed by design, offering options to reinstate unmodified 2′-hydroxyls and RNA functions.

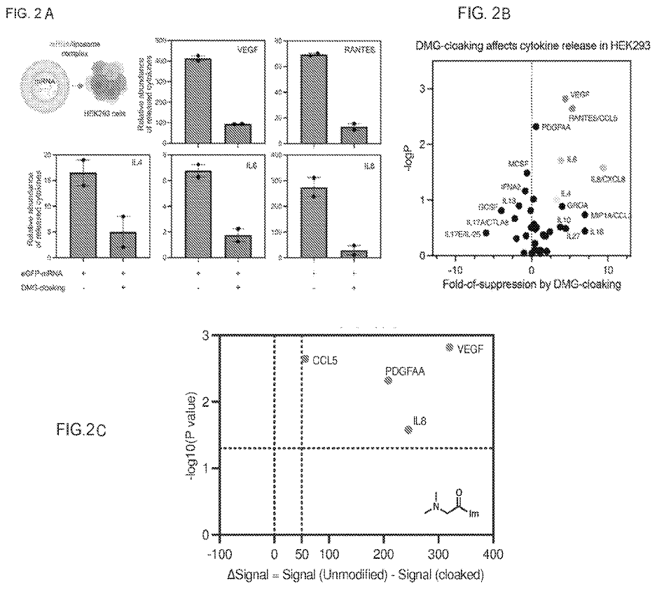

As an initial assessment of how cloaking affects the innate immune responses of mRNA, we measured the release of cytokines and immune activation markers from HEK293 cells that were lipofected by eGFP-mRNA with or without cloaking. HEK293 cells were transfected with 2 μg of mRNA with lipofectamine MessengerMax. After six hours post-transfection, analysis by a Luminex human 48-plex kit of released immune molecules showed a cloaking-dependent strong reduction in proinflammatory immune responses (FIG. 2); for instance, transfection with unmodified eGFP-mRNA led to a substantially enhanced release of proinflammatory chemokines and cytokines, including VEGF, RANTES/CCL5, PDGFAA, and IL8. In comparison. HEK293 cells that were transfected with intermediate cloaking (~50% of unpaired 2′-hydroxyls) of eGFP-mRNA significantly reduced the release of the above proinflammatory cytokines and activation by up to ~90%. The translation of eGFP-mRNA was insignificant at this time (6 hours post-transfection), so the release of these immunomodulatory cytokines/chemokines is largely due to cells sensing the mRNA. In addition, the expression of many mRNA-stimulated cytokines was not significantly modulated by cloaking, suggesting cloaking may selectively modulate specific innate immune responsive pathways.

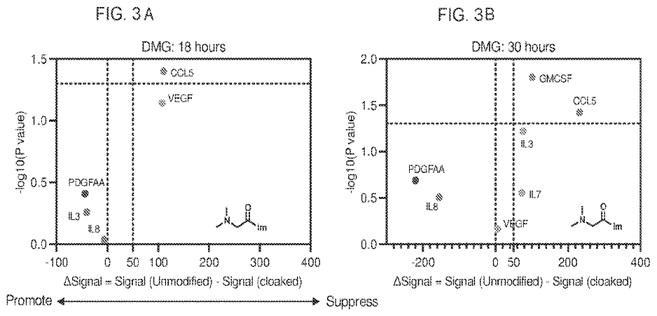

Transfection of in vitro transcribed RNA can trigger a potent Type I innate immune response in HEK293 cells (PMID: 30011268). Next, we investigated whether cloaking may modulate such response in HEK293 cells. To do this, HEK293 cells were lipofected by eGFP-mRNA with or without cloaking by DMG-Im. We measured the mRNA expression level of ISG15, an activation marker downstream of Type I innate immune response via the RIG-1 pathway. RT-qPCR showed that unmodified eGFP-mRNA moderately enhanced the expression of ISG15, while cloaking fully suppressed this activation (FIG. 3).

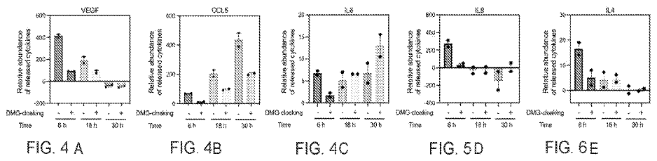

We further evaluated the effect of 2′-acylation on long-term immune response with Luminex human 48-plex assays in HEK293 cells that were transfected with modified eGFP-mRNA (FIG. 3 and FIG. 4). We found the release of proinflammatory Chemokine ligand 5 (CCL5) and cytokine Granulocyte-macrophage colony-stimulating factor (GMCSF) was increasingly suppressed overtime. In contrast, the secretion of the proinflammatory cytokine, Vascular endothelial growth factor (VEGF), was increasingly less suppressed over time, suggesting cloaking may selectively modulate certain innate immune responsive pathways in the time-space.

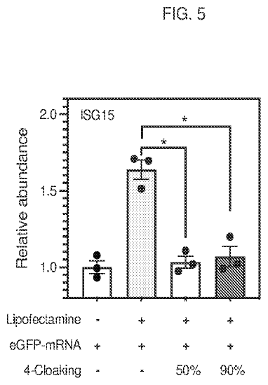

Transfection of in vitro transcribed RNA can trigger a potent Type I innate immune response in HEK293 cells (PMID: 30011268). Next, we investigated whether cloaking may modulate such response in HEK293 cells. To do this, HEK293 cells were lipofected by eGFP-mRNA with or without cloaking by DMG-lm. We measured the mRNA expression level of ISG15, an activation marker downstream of Type I innate immune response via the RIG-1 pathway. RT-qPCR showed that unmodified eGFP-mRNA moderately enhanced the expression of ISG15, while cloaking almost fully suppressed this activation (FIG. 5).

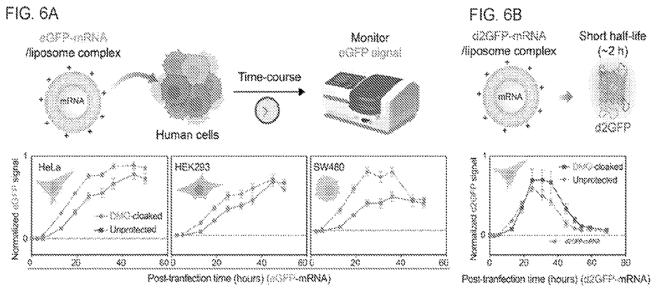

Recent studies have shown that 2′-acylation by some acyl groups can terminate translation if introduced in the coding region of mRNAs. Thus, we investigated whether adducts by N,N-dimethylglycine acylimidazole can spontaneously release within the cells. To explored this, we employed cloaked mRNAs encoding a green fluorescence protein (GFP) (FIG. 6). Our data showed that translation of GFP-mRNA with N,N-dimethylglycine adducts can be spontaneously restored in normal (e.g., HEK293) and cancerous cells (e.g., HeLa, SW480). Moreover, cloaking with DMG-Im extended the translational lifespan of an mRNA encoding a destabilized green fluorescent protein d2GFP by 31% in HeLa cells.

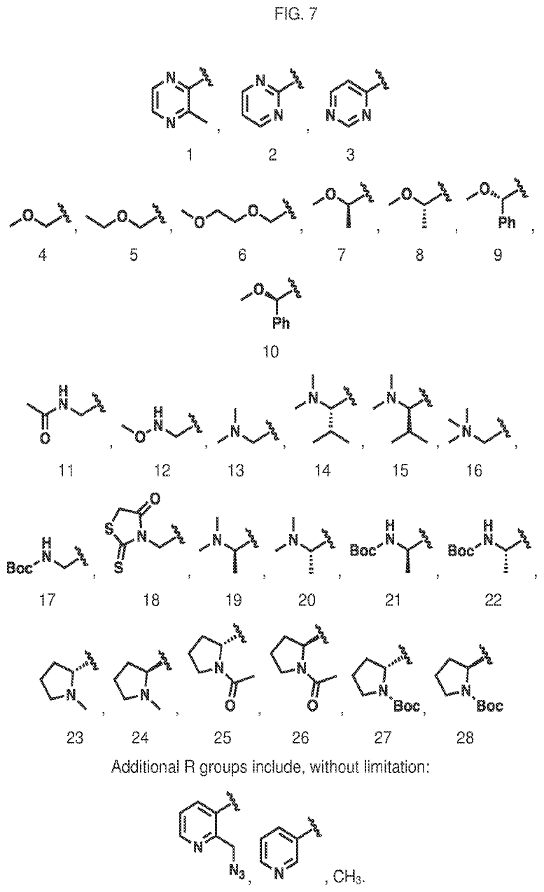

After confirming that the modified mRNAs can translate, we then investigated whether the variation of the chemical structures of acylating reagents may fine-tune RNA immunogenicity by differential modulation of innate immune response pathways. To this end, we assembled a panel of 28 acylating reagents containing structurally diversified substituents (FIG. 7). These reagents can be readily prepared with one-step activation of their corresponding low-cost carboxylic acids by 1,1′-carbonyldiimidazole (CDI). We paid attention to the installation of acyl groups with varied electrophilicity and sizes, which might later affect RNA interactions with cellular receptors/RNA sensors. Desired features of acyl adduct include sufficient chemical stability during RNA delivery, while being labile enough to restore mRNA translation efficiently. These structural features include aromaticity in reagents 1-3. We also installed a hetero atom (N or 0) at the alpha carbon to the carboxyl center of reagents 4-28 with varied steric bulk and charge status that may potentially modulate RNA interactions with cellular receptors and RNA sensors.

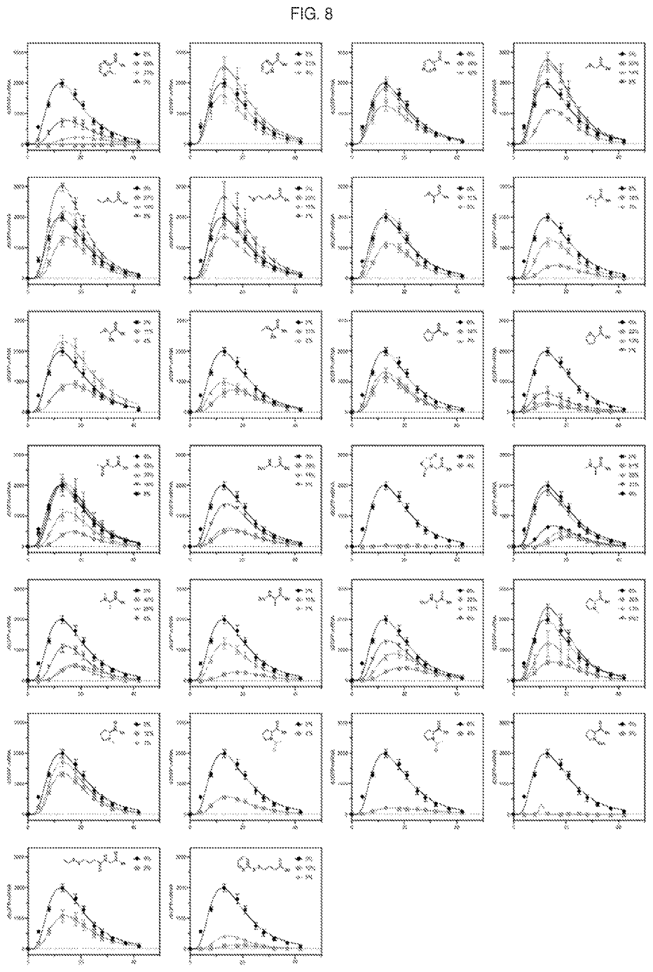

We screened these acylimidazole reagents for spontaneous restoration of mRNA translation in HEK293 cells by transfection with d2GFP-mRNA aliquots that were acylated with these acylimidazole reagents at various levels of 2′-modification. We monitored the green fluorescence signal in HEK293 cells over three days. Representative translation kinetics data are shown in FIG. 8 for d2GFP-mRNA cloaked with 1-28, demonstrating that a group of acylimidazole reagents allowed spontaneous RNA uncloaking in human cells to restore mRNA translation (FIG. 8). The chemical structure of acylimidazole reagents profoundly affects the spontaneous RNA uncloaking in human cells, as the geometry and electrophilicity of acyl group appear to modulate the translation efficiency, translation duration, and total protein output. Thus, we showed that acylation reagents are chemically tunable to alter properties. The data also show that acyl groups added to RNAs to reduce unwanted immune activation do not necessarily reduce translation, and can potentially enhance it.

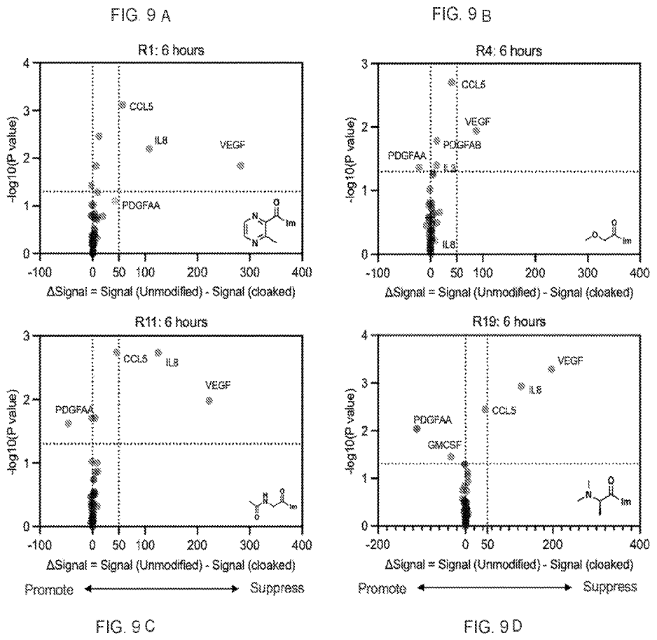

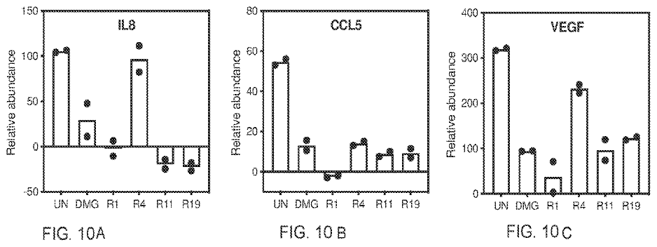

We next investigated whether the chemical structure of acylating reagents may differentially modulate certain innate immune response pathways. Among the reagents that allowed spontaneous restoration of mRNA translation, we further evaluated how four structurally diverse reagents R1, R4, R11, and R19 affect innate immune response (FIG. 9). Because the maximum level of cloaking varied among these reagents, we proceeded to equimolarly cloak at ~50% of unpaired accessible 2′-hydroxyls of eGFP-mRNA with a similar level of modification. We measured the release of cytokines and immune activation markers from HEK293 cells that were lipofected by modified eGFP-mRNA with or without cloaking. HEK293 cells were transfected with 2 μg of mRNA aliquots with lipofectamine MessengerMax. We observed that acylating reagents surprisingly demonstrated a chemical structure-dependent reduction in proinflammatory immune responses (FIG. 9); for instance, acylating reagents with an aromatic acyl group (R1) reduced the release of proinflammatory cytokines IL8, CCL5, and VEGF the most. In contrast, the acylating reagent with alpha alkoxy substituent (R4) only sufficiently suppressed the release of CCL5 rather than IL8 and VEGF, strongly suggesting structure-dependent modulation of certain innate immune-responsive pathways (FIG. 10). Structure features such as positive charges at the beta atom to the carboxyl group and additional steric bulk at alpha carbon did not appear to affect the immunogenicity of the underlying eGFP-mRNA greatly. Thus, acylating reagents with diverse chemical structures can differentially modulate certain innate immune-responsive pathways.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method for reducing innate immune responses by a cell in response to introduction of an exogenous RNA, the method comprising:
cloaking the RNA by acylating at least a portion of ribose in the RNA at the 2′ OH position with a 2′ RNA cloaking reagent.

2. The method of claim 1, wherein the cloaking reagent has the structure: 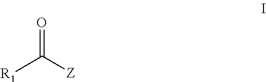
where R₁ is a substituted or unsubstituted alkyl group, a substituted or unsubstituted heteroalkyl group, a substituted or unsubstituted aryl or heteroaryl, a substituted or unsubstituted cycloalkyl; and
Z is selected from imidazole; 1,2,3-triazole, 1,2,4-triazole, azide, cyanide, anhydride, fluoride, and NHS ester.

3. The method of claim 2, wherein Z is imidazole.

4. The method of claim 2 or claim 3, wherein R₁ is selected from 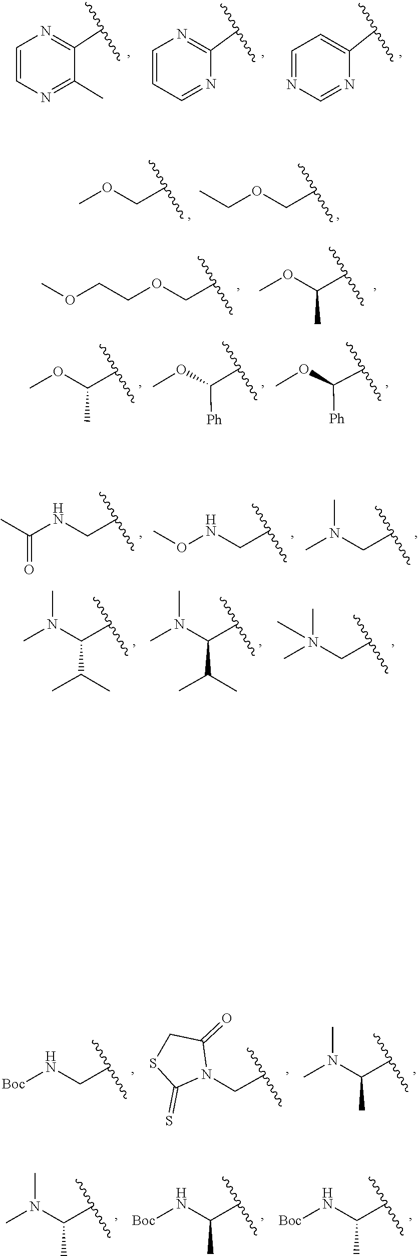 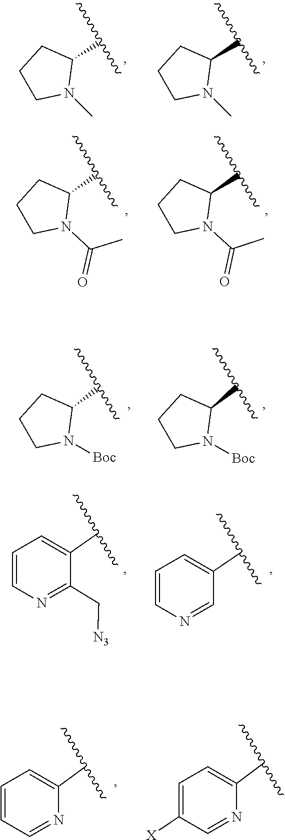
where X is Cl or Br, and CH₃.

5. The method of any of claims 2-4, wherein R1 is selected from

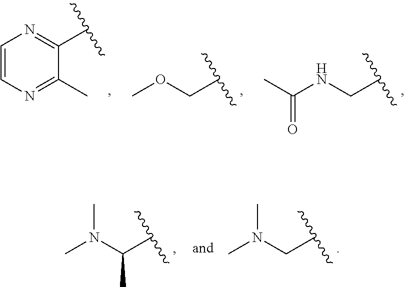

6. The method of claim 1, wherein the cloaking reagent has the structure: 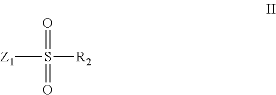
where R₂ is a substituted or unsubstituted alkyl group, a substituted or unsubstituted heteroalkyl group, a substituted or unsubstituted aryl or heteroaryl, a substituted or unsubstituted cycloalkyl; and
Z₁ is imidazole, 1,2,3-triazole, or 1,2,4-triazole.

7. The method of claim 6, wherein R₂ is selected from: 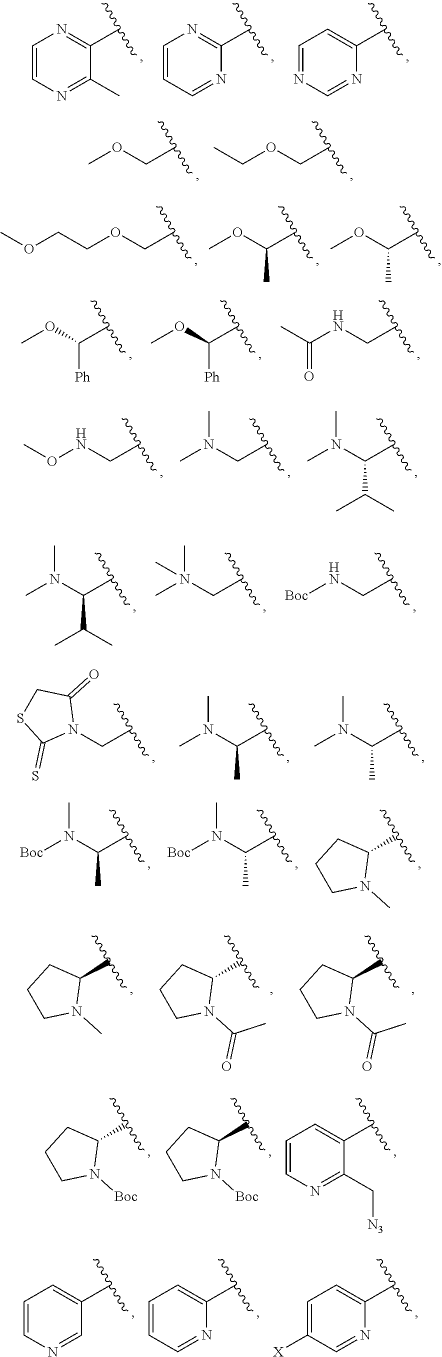
where X is CI or Br, and CH₃.

8. The method of any of claims 1-7, wherein the RNA is mRNA.

9. The method of claim 8, wherein the mRNA encodes a protein antigen.

10. The method of claim 8, wherein the antigen is a pathogen antigen, or a tumor-associated antigen.

11. The method of claim 8, wherein the mRNA encodes a therapeutic protein.

12. The method of any of claims 1-11, wherein the RNA further comprises unnatural modified nucleobases.

13. The method of any of claims 1-12, wherein after cloaking the RNA comprises at least 10% acylated 2′-OH.

14. The method of any of claims 1-13, wherein the RNA is formulated with a carrier for in vivo delivery to a cell.

15. The method of claim 14, wherein the carrier is a lipid nanoparticle (LNP), a polymeric nanoparticle, a lipid carrier such as a lipidoid, a liposome, a lipoplex, a peptide carrier, a nanoparticle mimic, a nanotube, or a conjugate.

16. The method of any of claims 1-15, wherein the RNA is formulated in a unit dosage form.

17. An RNA composition formulated for delivery to a cell, comprising:
RNA acylated in at least a portion of ribose at the 2′ OH position, wherein the acylated RNA has reduced innate immune responses by a cell in response to introduction of the RNA, relative to a non-acylated RNA.

18. The RNA composition of claim 17, wherein the acylation adduct is selected from 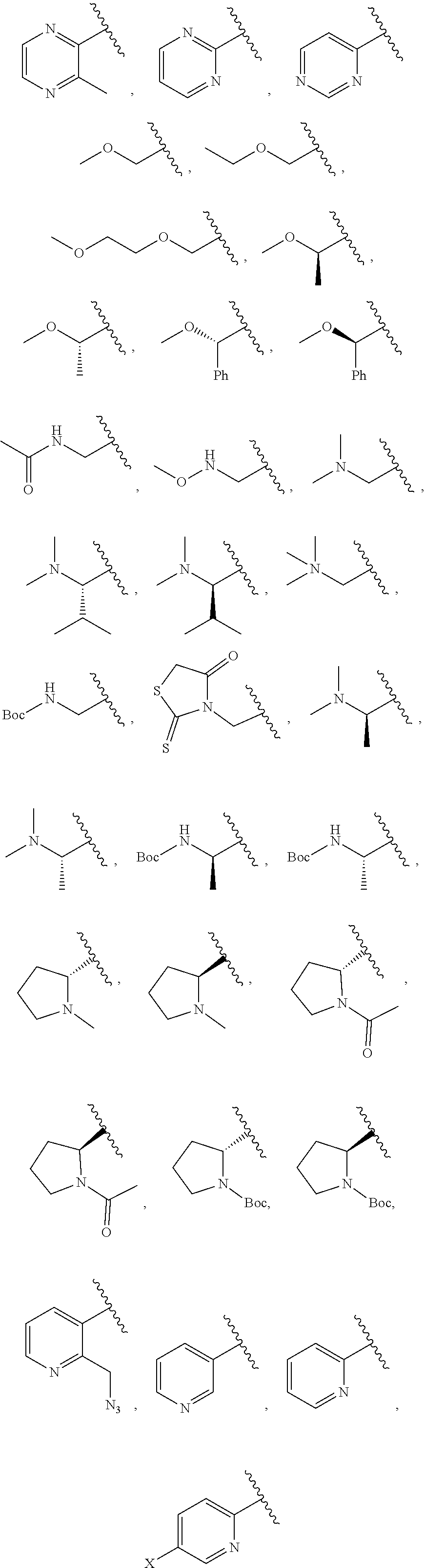
where X is Cl or Br, and CH₃.

19. The RNA composition of claim 17 or 18, wherein R1 is selected from

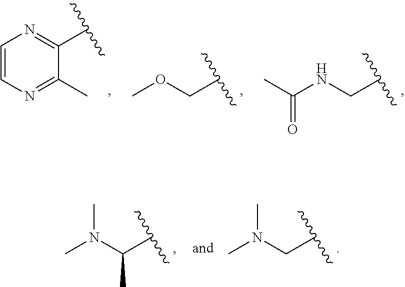

20. The composition of any of claims 17-19, wherein the RNA is mRNA.

21. The composition of any of claims 17-20, wherein the mRNA encodes a protein antigen.

22. The composition of claim 21, wherein the antigen is a pathogen antigen, or a tumor-associated antigen.

23. The composition of claim 21, wherein the mRNA encodes a therapeutic protein.

24. The composition of any of claims 17-23, wherein the RNA further comprises unnatural modified nucleobases.

25. The composition of any of claims 17-24, wherein after cloaking the RNA comprises at least 10% acylated 2′-OH.

26. The composition of any of claims 17-25, wherein the RNA is formulated with a carrier for in vivo delivery to a cell.

27. The composition of claim 26, wherein the carrier is a lipid nanoparticle (LNP), a polymeric nanoparticle, a lipid carrier such as a lipidoid, a liposome, a lipoplex, a peptide carrier, a nanoparticle mimic, a nanotube, or a conjugate.

28. The composition of any of claims 17-27, wherein the RNA is formulated in a unit dosage form.

29. A method for tuning the innate immune response of a cell to an exogenous RNA composition, the method comprising:
cloaking the RNA by 2′-hydroxyl acylation with 1 or more, usually 2 or more, 3 or more, 4 or more different 2′-OH cloaking reagents;
contacting a cell with the acylated RNA; and
measuring the release of innate immune mediators by the cell in response to the acylated RNA.

30. The method of claim 29 wherein the acylation adduct is selected from 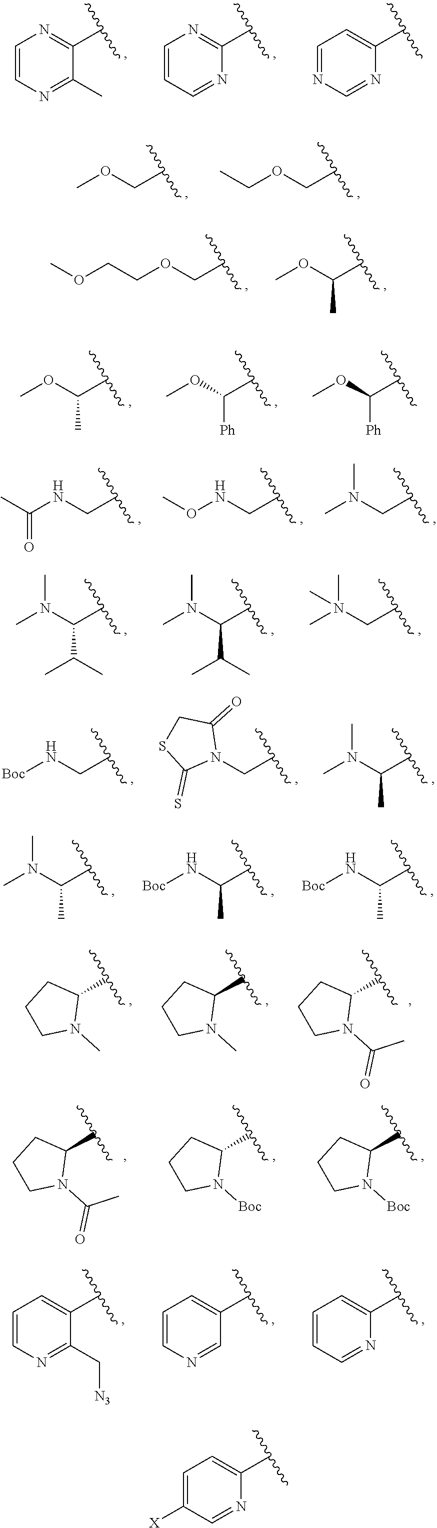
where X is Cl or Br, and CH₃.

31. The method of claim 29 or 30, wherein the acylation adduct is selected from

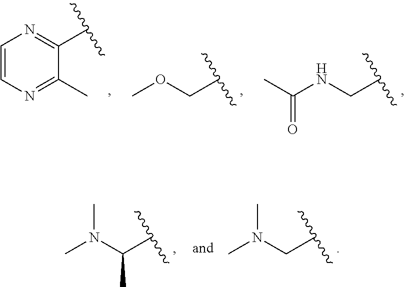

32. The method of any of claims 29-31, wherein the cell is a cell type targeted for expression of the RNA.

33. The method of any of claims 29-32, wherein the innate immune mediators comprise one or more of TNFα, IL-1β, IFNα, IFNβ, IL-6, CCL5, CXCL8, CXCL10, IL-8, IL-3, VEGF and PDGFAA.

34. The method of any of claims 29-33, wherein an acylation adduct is selected for use in cloaking based on the pattern of innate immune mediator release.
